(19) United States
(12) Patent Application Publication
(10) Pub. No.: US 2022/0348737 A1
(43) Pub. Date: Nov. 3, 2022

(54) PORUS SILICA-CONTAINING NANOPARTICLES, PRODUCTION METHOD THEREFOR, AND PHARMACEUTICAL COMPOSTION FOR RADIATION TREATMENT

(71) Applicants: KYOTO UNIVERSITY, Kyoto (JP); NATIONAL INSTITUTES FOR QUANTUM SCIENCE AND TECHNOLOGY, Chiba (JP); THE REGENTS OF THE UNIV. OF CALIFORNIA, Oakland, CA (US)

(72) Inventors: Fuyuhiko TAMANOI, Kyoto-shi, Kyoto (JP); Kotaro MATSUMOTO, Kyoto-shi, Kyoto (JP); Hiroyuki SAITOH, Chiba-shi, Chiba (JP); Ayumi SENOUCHI, Chiba-shi, Chiba (JP); Toshiki TAJIMA, Irvine, CA (US)

(73) Assignees: KYOTO UNIVERSITY, Kyoto (JP); NATIONAL INSTITUTES FOR QUANTUM SCIENCE AND TECHNOLOGY, Chiba (JP); THE REGENTS OF THE UNIV. OF CALIFORNIA, Oakland, CA (US)

(21) Appl. No.: 17/763,397

(22) PCT Filed: Sep. 25, 2020
(86) PCT No.: PCT/JP2020/036367

§ 371 Date: Mar. 24, 2022

Related U.S. Application Data
(60) Provisional application No. 62/907,043, filed on Sep. 27, 2019.

(51) Int. Cl. C08K 3/36; A61K 33/18; A61K 41/00; A61K 33/242; A61K 33/243; A61K 33/38; A61P 35/00

(52) U.S. Cl. CPC C08K 3/36; A61K 33/18; A61K 41/00; A61K 33/242; A61K 33/243; A61K 33/38; A61P 35/00; C01P 2006/16

(57) ABSTRACT

The present invention provides: nanoparticles including a compound that includes porous silica and at least one high-atom selected from the group consisting of gadolinium atoms, iodine atoms, gold atoms, silver atoms, and platinum atoms; and a pharmaceutical composition for radiation treatment, useful for treatment of solid tumors, etc., and including these nanoparticles and a pharmaceutically acceptable carrier.

### TECHNICAL FIELD

The present invention is related to a nanoparticle and a method for preparing the same, and a pharmaceutical composition for radiotherapy using the same, wherein the nanoparticle of the present invention comprises a compound containing a porous silica; and at least one high-Z atom selected from a group consisting of a gadolinium atom, an iodine atom, a gold atom, a silver atom and a platinum atom.

The present invention is also related to a method for treating solid cancer, or inhibiting a growth or proliferation of solid cancer using the nanoparticle or the pharmaceutical composition for radiotherapy.

### BACKGROUND ART

Radiation therapies (in particular, X-ray therapy) have been widely used as one of the prominent methods for cancer treatment currently. However, since currently used X-rays contain a wide range of wavelengths, there is a problem that some X-rays are absorbed on skin surface and thereby are not easy to reach cancer tissues. Further, X-rays used for radiation therapy affect normal tissues, for example, there are problems that damages such as skin inflammation are caused by an absorption of X-rays on the surface of skin, and cells in front of target tissues are damaged.

On the other hand, an approach of applying an Auger electron to cancer treatment is being studied, in which the Auger electron is emitted from the K-shell of the high-Z atom by the Auger effect which is provided by irradiating the high-Z atom with X-rays of specific energy (Non-Patent Documents 1 and 2). This approach is called photon activation therapy (PAT) and has been studied for its effects such as DNA damage and cell damage. Unlike usual X-rays (referred to as continuous X-rays or white X-rays), the X-ray of specific energy has a narrow energy range, and thus it is expected to have little effect on human body, and it is less likely to cause unintended secondary and concomitant effects. That is, the X-ray of specific energy may not have an adverse effect on normal cells. However, there are still no detailed reports on the effects of cell damage by using the Auger effect.

Here, some attempts have been made to irradiate nanoparticles with rays such as radiation to use the effects provided from the nanoparticles for medical application such as therapy (Patent Documents 1 to 5). However, no attempt is found to induce the Auger effect by using X-ray having a specific narrow range of energy.

### CITATION LIST

### Patent Document

Patent Document 1: JP 2012-524795 A
Patent Document 2: JP 2008-528114 A
Patent Document 3: JP 2011-524866 A
Patent Document 4: JP 2017-538783 A
Patent Document 5: JP 2007-536356 A

### Non-Patent Document

Non-Patent Document 1: Martin, R. F. & Feinendegen, L. E. The quest to exploit the Auger effect in cancer radiotherapy—a reflective review. Int. J. Rad. Biol. 92, 617-632 (2016).
Non-Patent Document 2: Yokoya, A. & Ito, T. Photon-induced Auger effect in biological systems. Int. J. Rad. Biol. 93, 743-756 (2017).

### SUMMARY OF THE INVENTION

### Problems to be Solved by Invention

An object of the present invention is to provide a nanoparticle and a method for preparing the same, and a pharmaceutical composition for radiotherapy using the same, each suitable for radiotherapy of solid cancer. Also, another object of the present invention is to provide methods such as a method for treating solid cancer, or inhibiting the growth or proliferation of solid cancer using the nanoparticles or the pharmaceutical composition for radiotherapy.

### Means to Solve Problems

The present invention includes, but is not limited to, the following embodiments.

[1] A nanoparticle comprising a compound which comprises
a porous silica; and
at least one high-Z atom selected from a group consisting of a gadolinium atom, an iodine atom, a gold atom, a silver atom and a platinum atom.
   (hereinafter, which may also be referred to as “nanoparticle(s) of the present invention” or “present nanoparticle(s)”).
   [2] The nanoparticle according to [1], wherein the high-Z atom is present on a surface of or inside the porous silica.
   [3] The nanoparticle according to [1] or [2], wherein the high-Z atom or a group containing the high-Z atom is chemically bound to the porous silica.
   [4] The nanoparticle according to any one of [1] to [3], wherein the porous silica contains a binding group which is bound to the high-Z atom or the group containing the high-Z atom.
   [5] The nanoparticle according to [4], wherein the binding group which is bound to the high-Z atom or the group containing the high-Z atom is selected from an amino group, a carboxy group, or a hydroxy group.
   [6] The nanoparticle according to any one of [1] to [5], wherein the porous silica contains a group for inhibiting an aggregation of nanoparticles.
   [7] The nanoparticle according to [6], wherein the group for inhibiting the aggregation of nanoparticles is selected from a phosphonate group, a sulfonate group, or a carboxylate group.
   [8] The nanoparticle according to any one of [1] to [7], wherein the nanoparticle has a particle size of 40 to 400 nm in diameter.
   [9] The nanoparticle according to any one of [1] to [8], wherein the porous silica is a mesoporous silica.
   [10] The nanoparticle according to [9], wherein the mesoporous silica is a biodegradable mesoporous silica.
   [10-1] The nanoparticle according to any one of [1] to [10] for X-ray irradiation.
   [11] The nanoparticle according to any one of [1] to [10], wherein the nanoparticle is subjected to an irradiation with X-ray capable of exciting a K-shell electron of the high-Z atom.
   [12] The nanoparticle according to [10-1] or [11], wherein the X-ray is monochromatic X-ray or characteristic X-ray.
   [13] A pharmaceutical composition for radiotherapy which comprises the nanoparticle according to any one of [1] to [12] and a pharmaceutically acceptable carrier.
   (hereinafter, which may also be referred to as “the pharmaceutical composition of the present invention for radiotherapy” or “the present pharmaceutical composition for radiotherapy”).
   [14] The pharmaceutical composition for radiotherapy according to [13], wherein the pharmaceutical composition is subjected to an irradiation with X-ray that is a radiation for irradiation.
   [15] The pharmaceutical composition for radiotherapy according to [13] or [14], wherein the pharmaceutical composition is used for treating solid cancer, or inhibiting a growth or proliferation of solid cancer.
   [16] The pharmaceutical composition for radiotherapy according to [15], wherein the solid cancer is selected from a brain tumor, a lung cancer, an ovarian cancer, a digestive system cancer, an osteosarcoma, or a head and neck cancer.
   [17] A method for treating solid cancer or inhibiting a growth or proliferation of solid cancer, wherein the method comprises subjecting the nanoparticle according to any one of [1] to [12] or the pharmaceutical composition for radiotherapy according to any one of [13] to [16] which is taken into a body of subject to a X-ray irradiation to destroy a cancer cell.
   (hereinafter, which may also be referred to as “the method of the present invention” or “the present method”).
   [18] A method for preparing the nanoparticle according to any one of [1] to [12], which comprises at least one step selected from
(a) a step of adding a substance containing iodine atom to a precursor substance for forming porous silica to incorporate the iodine atom inside a structure of the porous silica; and
(b) a step of adding a substance which contains a binding group for binding to a compound containing gadolinium atom or a precursor group of the binding group to a precursor substance for forming porous silica such that the porous silica can contain the binding group for binding to the compound containing gadolinium atom or the precursor group of the binding group; and then chemically bonding the compound containing gadolinium atom to the porous silica via the binding group for binding to the compound containing gadolinium atom or the precursor group of the binding group.
   (hereinafter, which may also be referred to as “the preparation method of the present invention” or “the present preparation method”).
   [18-1] A method for preparing the nanoparticle according to any one of [1] to [12], which comprises
a step of adding a substance which contains iodine atom and a substance which contains a binding group for binding to a compound containing gadolinium atom or a precursor group of the binding group to a precursor substance for forming porous silica such that the iodine atom can be incorporated inside a structure of the porous silica and the porous silica can contain the binding group for binding to the compound containing gadolinium atom or the precursor group of the binding group; and then chemically bonding the compound containing gadolinium atom to the porous silica via the binding group for binding to the compound containing gadolinium atom or the precursor group of the binding group.
   [19] The nanoparticle according to [11] or [12], the pharmaceutical composition for radiotherapy according to [14], or the method according to [17], wherein the X-ray is X-ray which has a spectrum peak within a range of E−0.03 keV or more and E+0.5 keV or less, with respect to the K-shell electron excitation energy E of the high-Z atom.
   [20] The nanoparticle according to [11] or [12], the pharmaceutical composition for radiotherapy according to [14], or the method according to [17], wherein
the X-ray is monochromatic X-ray having energy of 50.25 keV or 50.40 keV, and the high-Z atom is the gadolinium atom; or
the X-ray is monochromatic X-ray having energy of 33.20 keV, and the high-Z atom is the iodine atom.
   [21] The nanoparticle according to [11] or [12], the pharmaceutical composition for radiotherapy according to [14], or the method according to [17], wherein the X-ray is monochromatic X-ray or characteristic X-ray, which has an energy and/or wavelength capable of exciting the K-shell electron of the high-Z atom.
   [22] The nanoparticle according to any one of [1] to [12], or the pharmaceutical composition for radiotherapy according to any one of [13] to [15], for use in treating solid cancer or inhibiting a growth or proliferation of solid cancer.
   [23] A use of the nanoparticle according to any one of [1] to [12], or the pharmaceutical composition for radiotherapy according to any one of [13] to [15], in preparing a medicament for treating solid cancer or inhibiting a growth or proliferation of solid cancer.

### Effect of Invention

According to the present invention, it has become possible to provide nanoparticles and a method for preparing the same, and a pharmaceutical composition for radiotherapy using the same, each suitable for radiotherapy of solid cancer, wherein the nanoparticle comprises the compound containing porous silica; and at least one high-Z atom selected from the group consisting of a gadolinium atom, an iodine atom, a gold atom, a silver atom and a platinum atom. Further, according to the present invention, it has become possible to provide methods such as a method for treating solid cancer or inhibiting the growth or proliferation of solid cancer using the nanoparticles or the pharmaceutical composition for radiotherapy.

### BRIEF DESCRIPTION OF THE DRAWINGS

FIG. 1 shows a SEM image of the nanoparticles of Example 1 (Gd-MSN).

FIG. 2 shows a TEM image of the nanoparticles of Example 1 (Gd-MSN).

FIG. 3 shows a measurement result of FT-IR of the nanoparticles of Example 1 (Gd-MSN).

FIG. 4 shows a measurement result of thermal gravitational analysis (TGA) of the nanoparticles of Example 1 (Gd-MSN).

FIG. 5 shows a result of nitrogen adsorption/desorption measurement of the nanoparticles of Example 1 (Gd-MSN).

FIG. 6 shows a result of pore size distribution of the nanoparticles of Example 1 (Gd-MSN).

FIG. 7 shows a measurement result of zeta potential of the nanoparticles of Example 1 (Gd-MSN).

FIG. 8 shows STEM-EDX mapping images of the nanoparticles of Example 1 (Gd-MSN) and the nanoparticles of Comparative Example 1 (MSN).

FIG. 9 shows a STEM-EDX image of the nanoparticles of Example 1 (Gd-MSN) after sonication.

FIG. 10 shows a result of microscopic observation on cancer cells treated with the nanoparticles of Example 1 (Gd-MSN).

FIG. 11 shows a result of cytotoxicity test of the nanoparticles of Example 1 (Gd-MSN).

FIG. 12 shows a result of microscopic observation on tumor spheroids treated with the nanoparticles of Example 1 (Gd-MSN).

FIG. 13 shows a further result of microscopic observation on tumor spheroids treated with the nanoparticles of Example 1 (Gd-MSN).

FIG. 14 shows an outline of setup of a monochromatic X-ray irradiation device.

FIG. 15 shows an outline of spheroid sample place in the monochromatic X-ray irradiation device.

FIG. 16 shows X-ray absorption profile of gadolinium.

FIG. 17 shows a result (difference of irradiation time) of microscopic observation on tumor spheroids after monochromatic X-ray irradiation, where the tumor spheroids were treated with the nanoparticles of Example 1 (Gd-MSN) or the nanoparticles of Comparative Example 1 (MSN).

FIG. 18 shows a result (changes over time in cell destruction) of microscopic observation on tumor spheroids after monochromatic X-ray irradiation, where the tumor spheroids were treated with the nanoparticles of Example 1 (Gd-MSN).

FIG. 19 shows a result (difference of amounts of Gd-MSN) of microscopic observation on tumor spheroids after monochromatic X-ray irradiation, where the tumor spheroids were treated with the nanoparticles of Example 1 (Gd-MSN).

FIG. 20 shows a result (difference of energy of monochromatic X-ray) of microscopic observation on tumor spheroids after monochromatic X-ray irradiation, where the tumor spheroids were treated with the nanoparticles of Example 1 (Gd-MSN) or the nanoparticles of Comparative Example 1 (MSN).

### MODE FOR CARRYING OUT THE INVENTION

### Nanoparticle

According to one aspect of the present invention, the present invention is related to a certain specific nanoparticle. The nanoparticle of the present invention comprises a compound which contains a porous silica; and at least one high-Z atom selected from a group consisting of a gadolinium atom, an iodine atom, a gold atom, a silver atom and a platinum atom.

According to one embodiment of the present invention, the nanoparticle of the present invention may be a nanoparticle composed of the compound containing the porous silica and the high-Z atom.

Porous silica is a substance containing silicon dioxide (silica: SiO₂) as a main component and having a large number of pores. The porous silica may be in the form of particle. The porous silica may constitute the main component in the components constituting the nanoparticle of the present invention. The porous silica has an increased specific surface area due to the pores, and can load the high-Z atom efficiently on it. The porous silica may be a nano-molecule or a nano-polymer (nano-macromolecule). Nano-molecule means a nano-sized molecule, and nano-polymer means a nano-sized polymer (macromolecule). In the nano-molecule of porous silica, the molecule composed of silicon dioxide may constitute the porous silica. Further, the porous silica may be a nano-carrier. Nano-carrier means a nano-sized carrier. The nano-carrier of porous silica can serve as a support substance and/or substrate for supporting the high-atom. In the present specification, the nanosize usually refers to a size of 10 nm or more and 500 nm or less, and the preferable nanosize is a size of 40 nm or more and 400 nm or less.

The nanoparticle of the present invention contains the high-Z atom. The high-Z atom is one or more atoms selected from the group consisting of a gadolinium atom (Gd), an iodine atom (I), a gold atom (Au), a silver atom (Ag) and a platinum atom (Pt). The high-Z atom refers to an atom having a relatively large atomic number. In the present specification, the high-Z atom represents an atom having an atomic number of 47 or more. Specifically, the atomic numbers of the high-Z atom are 64 for Gd, 53 for I, 79 for Au, 47 for Ag, and 78 for Pt. The above-mentioned high-Z atoms have an advantage that it is easy to control X-ray capable of exciting the K-shell electron of atom and it is easy to obtain the Auger effect.

Here, “Auger effect” refers to a phenomenon that when an atom excited by energy transits to its original ground state, instead of emitting extra energy, the energy is given to an electron in the atom so as to emit the electron (See, for example, P. Auger: Sur les rayons β secondaires produits dans un gaz par des rayons X, C.R.A.S. 177 (1923) 169-171, and Auger, P. (1975) Surface Science 48, 1-8). Further, “Auger electron” refers to an electron emitted by the Auger effect. More specifically, a K-shell electron is emitted by X-ray irradiation, and then an electron is transferred from L-shell or M-shell thereto in order to fill in the space formed by the emission. The energy generated at this time is given to another electron and released. In the present invention, it is possible to use the Auger effect induced by the energy which excite the K-shell electron of the high-Z atom.

In the nanoparticles of the present invention, it is preferable that the high-Z atom is present on the surface of or inside the porous silica. In one preferred embodiment, the high-Z atom is present on the surface of the porous silica. In another preferred embodiment, the high-Z atom is present inside the porous silica. In another preferred embodiment, the high-Z atom is present both on the surface of and inside the porous silica. The presence of high-Z atom on the surface of or inside the porous silica facilitates the occurrence of Auger effect induced by X-ray irradiation.

The high-Z atom may be bound to the porous silica. In the case where the high-Z atom is bound to the porous silica, the high-Z atom can be strongly supported by the porous silica. In one preferred embodiment, the high-Z atom or the group containing high-Z atom is chemically bound to the porous silica. In the case where the bond between the high-atom and the porous silica is a chemical bond, the high-Z atom can be more stably bound. The form of bonding between the high-Z atom and the porous silica may be a form in which the high-Z atom is directly bound to the porous silica, or a form in which the high-Z atom is indirectly bound to the porous silica via other group(s). The form of chemical bond may be a covalent bond, an ionic bond, a metal bond, a hydrogen bond, or the like.

The porous silica may contain a binding group which is bound to the high-Z atom or the group containing the high-Z atom. In certain preferred embodiments, the porous silica contains the binding group which is bound to the high-Z atom or the group containing the high-Z atom. In these embodiments, the high-Z atom is bound to the porous silica, at least via the binding group in the porous silica. The binding group in the porous silica may be a functional group composed of component(s) other than silicon dioxide (SiO₂). The binding group is preferably an amino group, a carboxy group, or a hydroxy group, and more preferably an amino group. In embodiments where the porous silica contains the binding group, the high-Z atom is preferably a gadolinium atom. In this case, the bond between the porous silica and the high-atom can be easily obtained. For example, the gadolinium atom is preferably present as a part of gadopentetic acid (Gd(III) DTPA), and the gadopentetic acid may be bound to the amino group on the porous silica to introduce the Gd atom into the porous silica. Here, DTPA refers to diethylenetriaminepentaacetic acid. The chemical structure of gadopentetic acid is shown below.

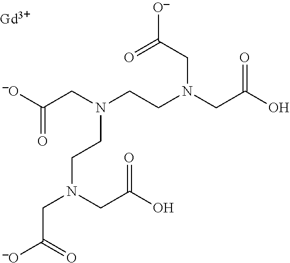 [SMILES: O=C([O-])CN(CCN(CC(=O)[O-])CC(=O)O)CCN(CC(=O)[O-])CC(=O)O]

In gadopentetic acid, Gd³⁺ and DTPA ion form a complex. Accordingly, the acid (non-ionized carboxylic acid) of DTPA and the amino group may be condensed, and the gadolinium atom in the nanoparticle may be present in an ionized form (specifically, Gd³⁺). The binding group is preferably present on the surface of the porous silica. The gadolinium atom is preferably present on the surface of the porous silica.

The porous silica may not contain the binding group which is bound to the high-Z atom or the group containing the high-Z atom. In certain preferred embodiments, the porous silica does not contain the binding group which is bound to the high-Z atom or the group containing the high-Z atom. The high-Z atom or the group containing the high-Z atom is directly bound to the porous silica.

In the case where the high-Z atom is an iodine atom, the porous silica may not contain the binding group. For example, the iodine atom is preferably present as a part of an iodized alkyl group, and the iodized alkyl group may be bound to silicon (Si) constituting the porous silica to introduce the iodine atom (I) into the porous silica. Examples of the iodized alkyl group include an iodized propyl group. The iodine atom can be introduced by using silane compounds which contain iodine atom as a starting substance for synthesizing porous silica. Accordingly, the iodine atom can be bound to the porous silica by a covalent bond. The iodine atom is preferably present inside the porous silica.

With respect to the high-Z atom, the gadolinium atom and the iodine atom are particularly preferable. The gadolinium atom can also be used for contrast agents of magnetic resonance imaging (MRI). Accordingly, the gadolinium atom can serve as both contrast agents and X-ray irradiation agents that provide the Auger effect. For example, cancer can be treated while the cancer is diagnosed. Whereas, the iodine atom has an advantage that the energy for exciting the K-shell electron is lower than that of other high-Z atoms, and X-ray having a lower energy can be used.

The nanoparticle of the present invention may contain two or more types of high-Z atoms. For example, the nanoparticle may contain both of gadolinium atom and iodine atom. Further, in certain preferred embodiments, one type of high-Z atom is present inside the porous silica, and another type of high-Z atom is present on the surface of the porous silica. Specifically, for example, in certain preferred embodiments, the iodine atom is present inside the porous silica and the gadolinium atom is present on the surface of the porous silica. As described above, for example, the iodine atom can be introduced into the porous silica by the iodized alkyl group, whereas the gadolinium atom can be introduced into the porous silica by bonding the gadopentetic acid to the amino group. In the case where the nanoparticle contains two or more types of high-Z atoms, the use value thereof may increase. For example, in the case where the nanoparticle contains both of the iodine atom and the gadolinium atom, the Auger effect can be provided by irradiating the iodine atom with X-ray while a contrast imaging is performed with the gadolinium atom. Thus, it becomes possible to carry out diagnosis and radiation therapy at the same time. The combination of two or more types of high-Z atoms is not limited to the combination of gadolinium atom and iodine atom. In the case where the nanoparticle contains two or more types of high-Z atoms, it is preferable that at least one of them is present on the surface of the porous silica and the other one of them is inside the porous silica.

The porous silica may contain a group which inhibits an aggregation of nanoparticles with each other (hereinafter, which may be referred to as “the group for inhibiting the aggregation of nanoparticles”). In certain preferred embodiments, the porous silica comprises the group for inhibiting the aggregation of nanoparticles. The nanoparticles might be prone to be aggregated, but the aggregation of the nanoparticles can be inhibited by the group for inhibiting the aggregation of nanoparticles. If the nanoparticles are aggregated, various disadvantages such as decreased uptake into cells and difficulty in formulation might come out. However such disadvantages can be reduced or eliminated by using the group for inhibiting the aggregation of nanoparticles. The group for inhibiting the aggregation of nanoparticles is preferably present on the surface of the porous silica. Accordingly, the aggregation can be efficiently inhibited. Examples of the group for inhibiting the aggregation of nanoparticles include, but are not limited to, a phosphonate group, a sulfonate group, or a carboxylate group, and preferably include a phosphonate group. These groups for inhibiting the aggregation of nanoparticles can efficiently inhibit the aggregation of nanoparticles. For example, the surface of nanoparticles can be negatively charged to inhibit the aggregation and further enhance the dispersibility. In another aspect, the porous silica does not contain the group for inhibiting the aggregation of nanoparticles.

The nanoparticle of the present invention preferably has a particle size of 40 to 400 nm in diameter. Such particle size facilitates the uptake of nanoparticles into cells. The particle size herein can be measured by observation with an electron microscopy (preferably, a transmission electron microscopy (TEM)). Here, with respect to the particle size of one particle, the length along the longest diagonal line of the particle is adopted. When the average particle size is needed, it can be determined based on an average particle size of a predetermined number (for example, 100) of nanoparticles. The particle size of nanoparticles is more preferably within a range of 50 to 300 nm in diameter. Thus, the average particle size of the nanoparticles is preferably within a range of 40 to 400 nm, and more preferably 50 to 300 nm.

In one preferred embodiment, the porous silica is a mesoporous silica. The mesoporous silica contains a large number of pores having a pore size (a pore diameter) usually of 2 to 50 nm. The mesoporous silica has an increased specific surface area, and accordingly the high Z atom can be loaded more efficiently. In addition, the mesoporous silica has an advantage that it is easily taken up into cells, as described later. Unless otherwise described, every term of the porous silica mentioned herein can be replaced with the mesoporous silica.

In certain preferred embodiments, the mesoporous silica may be a biodegradable mesoporous silica. The biodegradable mesoporous silica can be degraded in vivo over time. Examples of mechanism of decomposition include an enzymatic reaction. In the case of the biodegradable mesoporous silica, the nanoparticle can be degraded in the body to evacuate the resulting components, which makes it possible to carry out treatment and the like more safely. The biodegradable mesoporous silica can be obtained by using silane compounds having a biodegradable structure as a starting substance for synthesizing mesoporous silica. Examples of the biodegradable structure include a bond represented by S—S and/or S—S—S—S. For example, bis[3-(triethoxysilyl)propyl]tetrasulfide is a silane compound having an S—S—S—S bond between two Sis, and when this compound is incorporated into the structure of mesoporous silica, the structure containing the S—S—S—S bond between two Sis can be formed in the mesoporous silica. The bonds such as S—S and S—S—S—S have a relatively weak binding force, and are easily biodegraded.

The ratio of the high-Z atom to the porous silica (high-atom/porous silica) is not particularly limited, but may be, for example, within a range of 0.001 to 1 by weight, and further this ratio is preferably 0.01 to 0.5, and more preferably 0.05 to 0.2. This ratio (high-Z atom/porous silica) is particularly preferably 0.08 or more. Here, the weight amount of the high-Z atom can be determined by analyzing the high-Z atom in the nanoparticle using inductively coupled plasma atomic emission spectroscopy (ICP-AES).

The ratio of the high-Z atoms to the porous silica (high-Z atoms/porous silica) may also be specified in terms of molar ratio, and the molar ratio (for example, Gd/Si) may be calculated from the above-mentioned weight ratio.

The nanoparticles of the present invention can be used for X-ray irradiation. The nanoparticles can preferably be subjected to an irradiation with X-ray capable of exciting the K-shell electron of the high-Z atom. The X-ray is preferably monochromatic X-ray or characteristic X-ray. Details of X-ray irradiation on nanoparticles are described later.

### Method for Preparing Nanoparticles

According to one aspect of the present invention, the present invention is related to a method for preparing the above-mentioned nanoparticles. In preferred embodiments, the method of the present invention for preparing the nanoparticle comprises at least one step selected from
(a) a step of adding a substance containing iodine atom to a precursor substance for forming porous silica to incorporate the iodine atom inside a structure of the porous silica; and
(b) a step of adding a substance which contains a binding group for binding to a compound containing gadolinium atom or a precursor group of the binding group to a precursor substance for forming porous silica such that the porous silica can contain the binding group for binding to the compound containing gadolinium atom or the precursor group of the binding group; and then chemically bonding the compound containing gadolinium atom to the porous silica via the binding group for binding to the compound containing gadolinium atom or the precursor group of the binding group.

At first, general syntheses of porous silica are described below.

Examples of precursor substances for forming the porous silica include silane compounds. The silane compound herein refers to a compound in which organic group(s) is/are bound to a silicon atom (Si). As the precursor substances, the substance which is known as a starting substance for synthesizing porous silica (in particular, mesoporous silica) can be used. The precursor substances are not particularly limited, and examples thereof include alkoxysilane, alkylalkoxysilane, and the like. The porous silica can be prepared by reacting the precursor substances to condense. Specifically, tetraalkoxysilane can be used as the precursor substances, and examples thereof include tetraethoxysilane (also known as tetraethyl orthosilicate, chemical formula: Si(OC₂H₅)₄, abbreviation: TEOS) and the like, but are not limited thereto.

Further, a silane compound into which a functional group providing some function (for example, amino group, phosphonate group, carboxy group, carboxy group, hydroxy group, sulfonate group, carboxylate group, etc.; preferably, amino group, phosphonate group, etc.) is introduced may also be used as the precursor substances. In this case, the porous silica modified with the functional group providing some function can be obtained.

Here, as used herein, “alkyl” usually refers to an aliphatic chain alkyl group (for example, C1-8 alkyl, preferably C1-6 alkyl, and more preferably C1-3 alkyl), examples thereof include methyl, ethyl, propyl (including n-propyl and isopropyl), butyl (including n-butyl, tert-butyl and sec-butyl), hexyl and heptyl.

As used herein, “alkoxy” usually refers to C1-8 alkyloxy, preferably C1-6 alkoxy, and more preferably C1-3 alkoxy, and examples thereof include methoxy, ethoxy, propoxy (including n-propoxy and isopropoxy), butoxy (including n-butoxy, tert-butoxy, and sec-butoxy), hexyloxy, and heptyloxy.

The porous silica can be commercially available, or can be prepared by known preparation methods (for example, sol-gel method). In the preparation of porous silica (particularly, mesoporous silica), a compound for forming pores (particularly, mesopores) (so-called templates, hereinafter, also referred to as “template compound”) can be usually used. When the template compound is mixed during the reaction of the above-mentioned precursor substances, silica in which the template compound is incorporated is produced. The template compound is then removed from the resulting silica, and thus, the places of template are replaced with cavities, and a plural of pores are generated in the silica to produce the porous silica.

The template compound is not particularly limited, but examples thereof include cetyltrimethylammonium bromide (CTAB, chemical formula: C₁₆H₃₃N(CH₃)₃Br) and cetyltrimethylammonium chloride (CTAC, chemical formula: C₁₆H₃₃N(CH₃)₃Cl), and the like.

The porous silica has a three-dimensional network structure in which a plural of —Si—O— are connected with each other.

The synthesis of porous silica can usually be carried out in solvents. As the solvents, water, organic solvent(s), and a mixture of two or more thereof may be used. Examples of the organic solvent include, but are not limited to, one or more solvents selected from methanol, ethanol, isopropanol, and the like.

The nanoparticles of the present invention can be prepared by a method obtained by modifying the above-mentioned general synthesis of porous silica to a method that goes through step (a) and/or step (b). The nanoparticles of the present invention can be prepared by the step (a) (without going through the step (b)), or by the step (b) (without going through the step (a)), or by the step including both of steps (a) and (b).

The step (a) is suitable for preparing nanoparticles containing iodine atom. In the step (a), a substance containing iodine atom can be used. The substance containing iodine atom is preferably, but is not limited to, an iodized silane compound. Examples of the iodized silane compound include an iodized alkylalkoxysilane compound. Specifically, for example, (3-iodopropyl)trimethoxysilane can be used, but the iodized silane compound is not limited thereto. The chemical structure of (3-iodopropyl)trimethoxysilane is shown below.

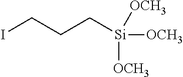 [SMILES: CO[Si](CCCI)(OC)OC]

As described above, the porous silica can be obtained by condensing the silane compounds (for example, TEOS) in the presence of template compound (for example, CTAB). In this reaction, the iodized silane compound (for example, (3-iodopropyl)trimethoxysilane) can be added. Thus, when the silane compounds which are precursor substance of porous silica are condensed, the iodized silane compound is incorporated into the structure in which the silane compounds are condensed. This is because the silane compounds bind each other (formation of —Si—O—Si— structure). In this manner, iodine atoms are incorporated inside the structure of the porous silica. For example, the iodine atom (I) may be bound to silicon (Si) in the porous silica via propylene group (—(CH₂)₃—).

Here, the iodized silane compound may be added in the middle of the condensation reaction of the silane compounds which are the precursor substance of porous silica. In this case, iodine atoms can be present at more increased amount in the outer portion of the porous silica than in the central portion. In the case where the iodine atom is present in the outer portion of the porous silica, Auger electron can be generated efficiently by X-ray irradiation.

The step (b) is suitable for preparing nanoparticles containing gadolinium atom. In the step (b), a compound containing gadolinium atom can be used. Examples of the compound containing gadolinium atom to be used include, but are not limited thereto, gadopentetic acid. Meanwhile, in the synthesis of the porous silica, a substance containing the binding group for binding to the compound containing gadolinium atom or the precursor group of the binding group is used. Examples of the group for binding to the compound containing gadolinium atom include, but are not limited thereto, an amino group (—NH₂). The amino group can form a bond with the acid moiety of gadopentetic acid by a condensation reaction (amidation). Here, the above-mentioned precursor group is a group which can be derivatized into the binding group for binding to the compound containing gadolinium atom. For example, the precursor group may be a group which can be derivatized into an amino group (for example, an amino group having a protecting group). Examples of the substance containing the binding group for binding to the compound containing gadolinium atom or the precursor group of the binding group include a silane compound containing amino group. Examples of the silane compound containing amino group include aminoalkyltrialkoxysilane. Specific examples thereof include, but are not limited to, 3-aminopropyltriethoxysilane. The chemical structure of 3-aminopropyltriethoxysilane is shown below.

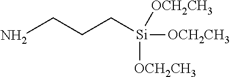 [SMILES: CCO[Si](CCCN)(OCC)OCC]

As described above, the porous silica can be obtained by condensing the silane compounds in the presence of the template compound. In this reaction, the silane compound (for example, 3-aminopropyltriethoxysilane) containing the binding group for binding to the compound containing gadolinium atom (for example, gadopentetic acid) or the precursor group of the binding group can be added. Thus, when the silane compounds which are precursor substance of porous silica are condensed, the above silane compound is incorporated into the structure in which the silane compounds are condensed. This is because the silane compounds bind each other (formation of —Si—O—Si— structure). In this manner, the porous silica can contain the binding group (for example, amino group) for binding to the compound containing gadolinium atom or the precursor group of the binding group.

Here, it is preferable to add the above-mentioned silane compound after allowing the condensation reaction of the silane compounds, which are precursor substance of the porous silica, to proceed to some extent. Thereby, the group capable of binding to the compound containing gadolinium atom can be present at increased amount on the outer surface of the porous silica. In this case, the compound containing gadolinium atom can be easily bound to the silane compound.

Then, the compound containing gadolinium atom is chemically bound to the porous silica via the binding group for binding to the compound containing gadolinium atom or the group derived from the above precursor group. Specifically, for example, in the case where the binding group for binding to the compound containing gadolinium atom or the group derived from the precursor group is an amino group, gadopentetic acid can be used, and the gadopentetic acid can be chemically bound to the amino group to bond the gadolinium atom to the porous silica.

Nanoparticles can also be prepared using both the step (a) and the step (b). In this case, nanoparticles containing both iodine atom and gadolinium atom can be obtained. For example, the substance containing iodine atom and the substance containing the binding group for binding to the compound containing gadolinium atom or the precursor group of the binding group may be added to the precursor substance for forming porous silica. Thus, the iodine atom can be incorporated in the structure of the porous silica, and the binding group for binding to the compound containing gadolinium atom or the precursor group of the binding group can be contained in the same porous silica. Then, the compound containing gadolinium atom can be chemically bound to the porous silica via the binding group for binding to the compound containing gadolinium atom or the group derived from the precursor group of the binding group. In the method where both the steps (a) and (b) are applied, the substances and compounds to be used in the reaction may be the same substances and compounds as described in each of the steps (a) and (b).

The step (a) is suitable for providing the presence of high-Z atoms inside the porous silica. On the other hand, the step (b) is suitable for providing the presence of high-atoms on the surface of the porous silica. Of course, the nanoparticles may also be prepared by methods other than the steps (a) and (b). For example, gold atom, silver atom and platinum atom may be incorporated into the porous silica in different methods from the method as described above. For example, with regard to the gold atom, the inventors have developed a method of bonding a binding substance containing gold cluster and protein to the surface of porous silica (Croissant, J G et al, Journal of Controlled Release 229 (2016) 183-191), and this method can be used. The inventors have also developed a method of incorporating iron oxide nanocrystals into porous silica nanoparticles (Liong, M et al., ACS NANO vol. 2 (2008), 889-896), and this method can be used in order to incorporate gold atom or silver atom (optionally, platinum atom) into nanoparticles. Of course, other methods may be used.

As described above, when the silane compound containing the functional group providing some function is added during the synthesis reaction of the porous silica, the porous silica containing the functional group providing some function can be prepared. According to this method, for example, the phosphonate group can be introduced into the porous silica by using the silane compound containing phosphonate group. Specifically, examples of the silane compound containing phosphonate group include, but are not limited to, 3-(trihydroxysilyl)propyl methylphosphonate monosodium. When the phosphonate group is introduced, the aggregation of nanoparticles can be inhibited. The chemical structure of 3-(trihydroxysilyl)propyl methylphosphonate monosodium is shown below.

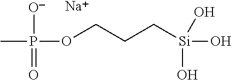

Here, it is preferable to add the silane compound containing phosphonate group after allowing the condensation reaction of the silane compounds, which are the precursor substance of porous silica, to proceed to some extent. Thereby, the phosphonate group can be present with an increased amount on the outer surface of the porous silica. In this case, the effect of inhibiting the aggregation of nanoparticles can be enhanced.

Further, the silane compound having the biodegradable structure may also be added during the synthesis reaction of porous silica (preferably, mesoporous silica). In this case, a biodegradable porous silica (preferably, a biodegradable mesoporous silica) can be prepared. Examples of the silane compound having the biodegradable structure include, but are not limited to, a compound having S—S or S—S—S—S bond in the molecule, for example, bis[3-(triethoxysilyl)propyl]tetrasulfide and the like. Bis[3-(triethoxysilyl)propyl]tetrasulfide has the structure represented by (C₂H₅O)₃—Si—C₃H₆—S—S—S—S—C₃H₆—Si—(C₂H₅O)₃ and contains S—S—S—S bond between the two Sis.

Also, a fluorescently labeled silane compound may be added during the synthesis reaction of porous silica (preferably, mesoporous silica). In this case, the fluorescently labeled porous silica (preferably, mesoporous silica) can be prepared. Specifically, for example, the labeling can be performed by using a fluorescently labeled compound such as rhodamine B isothiocyanate. The rhodamine B isothiocyanate can be introduced, for example, via amino group. When nanoparticles are fluorescently labeled, they are effective in biological experiments and the like.

### X-ray Irradiation to Nanoparticles and Destruction of Cancer Cells

According to one aspect of the present invention, the present invention is related to X-ray irradiation to the above-mentioned nanoparticles and the destruction of cancer cells which is caused by the X-ray irradiation. The nanoparticles of the present invention are suitable for X-ray irradiation. Specifically, the X-ray irradiation to target the high-Z atom can be performed. Due to the X-ray irradiation that can excite the K-shell electron of the high-atom, the Auger electron can be emitted from the high-Z atom.

In general, Auger electrons can damage DNA and other cellular components. The high-Z atoms as described above are suitable for emitting Auger electrons. However, an effective distance of Auger electron is limited, and there had been no thorough studies that sufficiently demonstrated the cell-destroying effect of Auger electron before the present invention. The present invention differs from previous studies and has an advantage in that the high-Z atom and the porous silica are combined.

The nanoparticles of the present invention are characterized in that they are easily taken up into a cell, in particular, a cancer cell. It is confirmed that when nanoparticles are brought into contact with a cell, the nanoparticles enter the cell. In accordance with one of hypothesis, cell uptake is due to the action of endocytosis mechanisms involving endosome vesicles, and these vesicle transportation can deliver nanoparticles to lysosomes located adjacent to the cell nucleus. Of course, the present invention is not limited by the above hypothesis. Thus, according to the nanoparticles of the present invention, the high-Z atom can be placed near the cell nucleus.

The nanoparticles of the present invention are also useful for targeting solid cancers such as tumors. When the nanoparticles are administrated to humans and animals, the nanoparticles can accumulate in the solid cancer. Accordingly, the nanoparticles of the present invention have at least two advantages of reaching solid cancer and being taken up into the cancer cells.

When the high-Z atom located near the cell nucleus is irradiated with X-rays capable of exciting the K-shell electron of the high-Z atom, Auger electron is emitted from the high-Z atom, and this electron can damage the cell. Near and around the cell nucleus, there are important cell functions including organelles, and which can be damaged by Auger electron. Accordingly, it is possible to damage cells efficiently and effectively. Then, cancer cells can be destroyed or killed due to this cell damage.

The X-rays that can excite the K-shell electron of the high-Z atom are different depending on each of the high-Z atoms, and each of the high-Z atoms has an individual energy level and/or wavelength. The X-ray may be an X-ray having an energy capable of exciting the K-shell electron. Further, the X-ray can be an X-ray having a wavelength capable of exciting the K-shell electron. According to “International tables for crystallography C, Table 4.2.2.4 theoretical calculations” presented by International Union of Crystallography (IUCr), the K-shell electron excitation wavelengths (the corresponding X-ray wavelengths) and the K-shell electron excitation energies of Gd, I, Au, Ag and Pt are shown in Table 1.

TABLE 1

| | λ (Å) | λ (nm) | E (keV) |
|---|---|---|---|
| Gd | 0.2467265 | 0.02467 | 50.25 |
| I | 0.3736775 | 0.03737 | 33.18 |
| Au | 0.1535699 | 0.01536 | 80.73 |
| Ag | 0.4857609 | 0.04858 | 25.52 |
| Pt | 0.1581346 | 0.01581 | 78.40 |

With respect to the gadolinium atom, X-ray having an energy of 50.25 keV is most suitable for exciting the K-shell electron. This is because the K-shell electron excitation energy of gadolinium atom is 50.25 keV. However, even if the energy level is not the best point, the K-shell electron may be excited, and it is confirmed that the effect can be exerted even by using X-ray having an energy of 50.40 keV for the gadolinium atom. Accordingly, when the gadolinium atom is irradiated with the X-ray having an energy of 50.25 keV or 50.40 keV, Auger electron can be emitted from the gadolinium atom. Here, the wavelength of X-ray that can excite the K-shell electron of the gadolinium atom is 0.02467 nm or 0.02460 nm.

With respect to the iodine atom, X-ray having an energy of 33.18 keV is suitable for exciting the K-shell electron. This is because the K-shell electron excitation energy of iodine atom is 33.18 keV. Accordingly, when the iodine atom is irradiated with the X-ray having an energy of 33.18 keV, Auger electron can be emitted from the iodine atom. Here, the wavelength of X-ray that can excite the K-shell electron of the iodine atom is 0.03737 nm.

Similarly with respect to the gold atom, the silver atom and the platinum atom, the X-ray energies (or wavelengths) suitable for exciting K-shell electron are 80.73 keV (or 0.01536 nm) for gold atom, 25.52 keV (or 0.04858 nm) for silver atom, and 78.40 keV (or 0.01581 nm) for platinum atom. Accordingly, when each of these high-Z atoms is irradiated with X-ray of each of these energies (or wavelengths), Auger electron can be emitted.

Here, the K-shell electron excitation energy is also referred to as the K-shell absorption edge energy, and the K-shell electron excitation wavelength is also referred to as the K-shell absorption edge wavelength.

The X-ray may be an X-ray having a spectrum peak at preferably E−0.5 keV or more, more preferably E−0.3 keV or more, still more preferably E−0.1 keV or more, and further more preferably E−0.05 keV or more, with respect to the K-shell electron excitation energy E of high-Z atom. The X-ray may be an X-ray having a spectrum peak at preferably E+0.8 keV or less, more preferably E+0.7 keV or less, still more preferably E+0.6 keV or less, further more preferably E+0.5 keV or less, with respect to the K-shell electron excitation energy E of high-Z atom. For example, the X-ray may be an X-ray which has the spectrum peak at any one of E−0.5 keV or more, E−0.3 keV or more, E−0.1 keV or more, or E−0.05 keV or more, and which has the spectrum peak at any one of E+0.8 keV or less, E+0.7 keV or less, E+0.6 keV or less, or E+0.5 keV or less, as described above. In particular, the X-ray is preferably an X-ray which has a spectrum peak within a range of E−0.03 keV or more and E+0.5 keV or less, with respect to the K-shell electron excitation energy E of high-Z atom. As described above, E is different depending on each of the high-Z atoms, and Auger electron can be efficiently emitted by irradiating with X-ray having energy corresponding to each of the high-Z atoms. Further, the Auger effect may be exhibited even in the case of an energy in the vicinity of the K-shell electron excitation energy E of high-Z atom. In particular, the Auger effect may be exhibited even in the case of an energy slightly higher than the K-shell electron excitation energy E. Accordingly, the energy is preferably an energy of E+0.5 keV or less as described above. On the other hand, if the energy is lower than the K-shell electron excitation energy E, it might not be easy to exhibit the Auger effect, and accordingly, the energy is preferably an energy of E−0.03 keV or more as described above. On the bases of calculation, it can be understood that the energy is preferably within a range of 50.22 to 50.75 keV for gadolinium atom (E=50.25 keV), and the energy is preferably within a range of 33.15 to 33.68 keV for iodine atom (E=33.18 keV). From the viewpoint of exhibiting the Auger effect, the X-ray spectrum peak is preferably E−0.02 keV or more, and more preferably E−0.01 keV or more. Further, the X-ray spectrum peak is preferably E+0.3 keV or less, and more preferably E+0.2 keV or less.

The X-ray is preferably monochromatic X-ray or characteristic X-ray. Further, the X-ray is more preferably monochromatic X-ray. Monochromatic X-ray refers to X-ray having an extremely narrow range of energy. When the term of “a monochromatic X-ray having an energy of E₁” is used herein, a spectrum peak of the monochromatic X-ray is at the position of the energy of E₁. For example, the monochromatic X-ray does not include an X-ray having an energy of E₁-ΔE keV or less, nor an X-ray having an energy E₁-ΔE keV and more. Here, it is provided that ΔE≤E×10⁻³. The characteristic X-ray is an X-ray in which excess energy is emitted as X-ray when an electron from the outer shell transits to the hole generated by the excitation of inner core. The energy of characteristic X-ray is a substance-specific value which is determined by the energy difference between the inner core and the outer shell. Accordingly, in this case, it would be difficult to extract monochromatic X-ray having arbitrary energy. Unlike continuous X-ray (or white X-ray), monochromatic X-ray and characteristic X-ray have a narrow energy range, so that the damage on normal cells and tissues which is caused by X-ray irradiation can be effectively reduced. In particular, monochromatic X-ray is excellent in such effects. The monochromatic X-rays can be extracted by monochromatizing a white X-ray generated in a synchrotron radiation facility or a white X-ray generated by an X-ray generator by means of a spectroscope (e.g. a monochromator). The characteristic X-ray can be extracted by characteristic X-ray-monochromatization of a white X-ray generated by the X-ray generator by means of a spectroscope. However, the method of generating monochromatic X-ray and characteristic X-ray is not limited to thereto.

As shown in Examples described below, when the nanoparticles containing gadolinium atom were irradiated with X-ray having an energy of 50.25 keV, cancer cells were able to be effectively destroyed. In addition, when the nanoparticles containing gadolinium atom were irradiated with X-ray having an energy of 50.40 keV, cancer cells were able to be destroyed, although the effect was lower than the energy of 50.25 keV was used. On the contrary, when the nanoparticles containing gadolinium atom were irradiated with X-ray having an energy of 50.00 keV, almost no cancer cell destruction occurred. The drastic difference in the effects of 50.25 KeV and 50.00 KeV in X-rays suggests the theories that Auger electrons exert the cell-destroy effect.

### Pharmaceutical Composition for Radiotherapy

According to one aspect of the present invention, the present invention is related to the pharmaceutical composition for radiotherapy which comprises the nanoparticles of the present invention.

The present pharmaceutical composition for radiotherapy comprises the nanoparticles of the present invention described above and a pharmaceutically acceptable carrier. The pharmaceutically acceptable carrier may be liquid or solid. The carrier may be excipient, diluent, auxiliary agent (adjuvant) or the like. Examples of the liquid carrier include water and organic solvents. Examples of the organic solvents include, but are not limited to, alcohol solvents such as methanol and ethanol, and the like. Examples of the solid carrier include lactose, crystalline cellulose, starch, and the like. The carriers described herein are merely examples, and known carriers can be appropriately used for the pharmaceutical composition for radiotherapy.

The pharmaceutical composition for radiotherapy may be subjected to an irradiation with X-rays as radiation for irradiation. As described above, when X-ray irradiation is performed, electrons can be emitted from the high-Z atoms in nanoparticles by the Auger effect. When the pharmaceutical composition for radiotherapy is used, the pharmaceutical composition for radiotherapy or nanoparticles in the composition can reach the target site easily.

The pharmaceutical composition for radiotherapy may be used for treating solid cancer, or inhibiting a growth or proliferation of solid cancer. As described above, Auger electrons emitted from the high-Z atoms by X-ray irradiation can destroy cancer cells. Thus, the pharmaceutical composition for radiotherapy is useful for treating solid cancer and inhibiting the growth or proliferation of solid cancer.

Examples of the solid cancers include, but are not limited to, a brain tumor, a lung cancer, an ovarian cancer, a digestive system cancer, an osteosarcoma, or a head and neck cancer.

The pharmaceutical composition for radiotherapy may be administrated by an appropriate administration method. The administration method may be oral administration or parenteral administration. Examples of parenteral administration include injection (intravenous injection, and subcutaneous injection, intramuscular injection, etc.), suppository administration, external application (skin application, mucosal application), and the like. The dose of the pharmaceutical composition for radiotherapy is not particularly limited, but is preferably an amount in which Auger effect is exhibited when the nanoparticles of the present invention are subject to the X-ray irradiation.

According to one aspect of the present invention, the present invention is related to a method for treating solid cancer or inhibiting a growth or proliferation of solid cancer. The method comprises subjecting the above-mentioned nanoparticles or the above-mentioned pharmaceutical composition for radiotherapy which is taken into a body of subject to the X-ray irradiation to destroy the cancer cell. As the X-ray, an X-ray capable of exciting the K-shell electron of the high-Z atom can be used. The mechanism that cancer cells are destroyed is as described above. The subject includes a patient. Naturally, the method can be applied to human beings, but it can also be applied to animals other than human beings.

The X-ray irradiation time may vary depending on the severity of the disease to be treated, the patient's tolerance for X-ray irradiation, and the like, but is not particularly limited, and examples thereof may include 1 minute or more, 2 minutes or more, 3 minutes or more, and 5 minutes or more. Further, the X-ray irradiation time is not particularly limited, but examples thereof may include 240 minutes or less, 180 minutes or less, 150 minutes or less, or 120 minutes or less. For example, the X-ray irradiation time may be 10 minutes, 30 minutes, 60 minutes, 90 minutes, or the like.

As shown in Examples described below, the nanoparticles of the present invention have excellent targeting properties for solid cancers, and can easily penetrate into the cancer cells. Then, when the nanoparticles are irradiated with X-rays, cancer cells can be destroyed by Auger electrons. Therefore, this method can effectively treat the solid cancers or effectively inhibit the growth or proliferation of solid cancers.

Here, nanoparticles containing high Z element are useful not only for treatment but also for diagnosis. For example, nanoparticles containing gadolinium atom can be used as an amplification substance for MRI. Thus, for example, the nanoparticles of the present invention can be used not only for the treatment of cancers but also for the diagnosis of cancers, and further can be applied for theranostics (medical technology that combines treatment and diagnosis). Accordingly, the present specification discloses a pharmaceutical composition for cancer diagnosis which comprises the above-mentioned nanoparticles, and a method for diagnosing cancer using nanoparticles or the pharmaceutical composition for cancer diagnosis. Further, the present specification discloses a pharmaceutical composition for diagnosis and treatment of cancer, and a method for diagnosing and treating cancer using nanoparticles or the pharmaceutical composition for cancer diagnosis. The pharmaceutical composition for cancer diagnosis and the pharmaceutical composition for diagnosis and treatment of cancer may have the same constitutes as the above-mentioned pharmaceutical composition for radiotherapy. Accordingly, these compositions may be replaced with each other.

### EXAMPLES

Hereinafter, the present invention is described in more detail with reference to Examples, however, these examples are merely for assisting the understanding of the present invention, and the present invention should not be limited to these examples.

### Measurement Device and Measurement Conditions

Measurement devices and measurement conditions for nanoparticles are as follows.

Scanning Electron Microscopy (SEM) was carried out with JEOL JSM-75FCT. Transmission Electron Microscopy (TEM) was carried out with JEOL JEM-2100F. The scanning transmission electron microscopy-energy dispersive X-ray (STEM-EDX) analysis was carried out with JEOL JEM-2200FS+JED2300T system operated at 200 kV. The quantitative measurement of the elements in the substances was performed by the Cliff-Lorimer ratio method to obtain the relative concentrations from the integrated EDX intensities. Zeta-potential measurement was carried out with ELS Z (Otsuka Electronics). Fourier transform infrared (FT-IR) spectra was measured with Bruker E400 FT-IR spectrometer using potassium bromide pellets. Thermal gravitational analysis (TGA) was measured using TA Instruments Q-500 thermal gravitational analyzer under airflow with a temperature gradient of 5° C./min. Low-pressure N₂ adsorption measurement at 77 K was carried out with Quantachrome Autosorb iQ volumetric gas adsorption analyzer in the conditions that ultrahigh-purity-grade N₂ and He (99.999% purity) (for estimation of dead space) were used. ICPE−9000 (Shimadzu) was used for ICP-AES analysis to determine the amount of gadolinium loaded onto MSN.

First, the nanoparticles obtained by the preparation method of the present invention and properties of the nanoparticles are described below.

### Example 1

### Preparation of Gadolinium-containing Mesoporous Silica Nanoparticles (Gd-MSN)

A mixture of cetyltrimethylammonium bromide (CTAB, 98%, Sigma-Aldrich) (250 mg), 8 M NaOH aqueous solution (219 μL) and water (120 mL) was vigorously stirred at 80° C. to prepare a CTAB solution. Whereas, rhodamine B isothiocyanate (RITC, Sigma-Aldrich) (2.5 mg) was dissolved in ethanol (5 mL), and 3-aminopropyltriethoxysilane (APTS, 99%, Wako) (6 μL) was added thereto, and the mixture was then mixed by stirring for 30 minutes at room temperature to prepare a RITC-APTS solution. Then, tetraethyl orthosilicate (TEOS, also known as tetraethoxysilane, 95%, Wako) (1.2 mL) and 3-aminopropyltriethoxysilane (APTS) (250 μL) were mixed with the above RITC-APTS solution, and then the mixture solution was added dropwise to the CTAB solution. The mixture was stirred for 15 minutes. To the mixture, 3-(trihydroxysilyl)propyl methylphosphonate monosodium aqueous solution (50%) (315 μL) was then added, and the mixture was stirred. The resulting solid product was collected by centrifugation, and washed three times with ethanol. The solid product was refluxed in a mixed solution of concentrated HCl solution (2.3 mL) and ethanol (60 mL). Accordingly, the CTAB as template was removed. Then, the solid product was centrifuged, washed twice with ethanol, and dried overnight. The solid product consisted of mesoporous silica nanoparticles modified with amino groups (MSN-NH₂). The solid product (10 mg) was added to 0.1 M gadopentetic acid solution (Gd(III)-DTPA, 97%, produced by TRC), and ultrasonically dispersed for 15 minutes. The resulting dispersed solution was stirred for 24 hours. Then, the solid product was collected by centrifugation, washed sequentially with water and ethanol to remove unreacted Gd(III)-DTPA, and dried overnight to obtain gadolinium-containing mesoporous silica nanoparticles (Gd-MSN).

Here, in the above preparation, the rhodamine B labeling was performed for analysis and experiments. However, it is naturally possible to omit the rhodamine B labeling to prepare gadolinium-containing nanoparticles (Gd-MSN).

### Comparative Example 1

### Preparation of Mesoporous Silica Nanoparticles (MSN)

For comparison with the nanoparticles of the present invention, mesoporous silica nanoparticles containing no high-Z atom were prepared as below.

A mixture of cetyltrimethylammonium bromide (CTAB, 98%, Sigma-Aldrich) (250 mg), 8 M NaOH aqueous solution (219 μL) and water (120 mL) was vigorously stirred at 80° C. to prepare a CTAB solution. Whereas, rhodamine B isothiocyanate (RITC, Sigma-Aldrich) (2.5 mg) was dissolved in ethanol (5 mL), and 3-aminopropyltriethoxysilane (APTS, 99%, Wako) (6 μL) was added thereto, and the mixture was then mixed by stirring for 30 minutes at room temperature to prepare a RITC-APTS solution. Then, tetraethyl orthosilicate (TEOS, also known as tetraethoxysilane, 95%, Wako) (1.2 mL) and 3-aminopropyltriethoxysilane (APTS) (250 μL) were mixed with the above RITC-APTS solution, and then the mixture solution was added dropwise to the CTAB solution. The mixture was stirred for 15 minutes. To the mixture, 3-(trihydroxysilyl)propyl methylphosphonate monosodium aqueous solution (50%) (315 μL) was then added, and the mixture was stirred. The resulting solid product was collected by centrifugation, and washed three times with ethanol. The solid product was refluxed in a mixed solution of concentrated HCl solution (2.3 mL) and ethanol (60 mL). Accordingly, the CTAB as template was removed. Then, the solid product was centrifuged, washed twice with ethanol, and dried overnight. As a result, mesoporous silica nanoparticles (MSN) were obtained.

### Example 2

### Properties of Nanoparticles of the Present Invention Prepared in Example 1

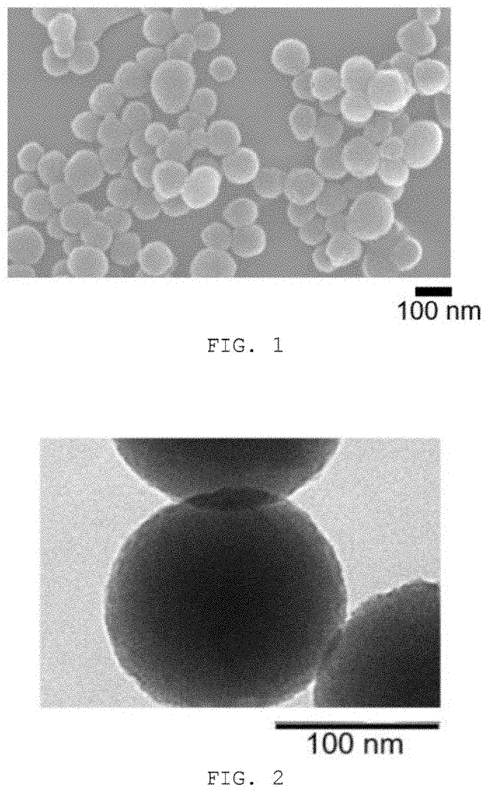

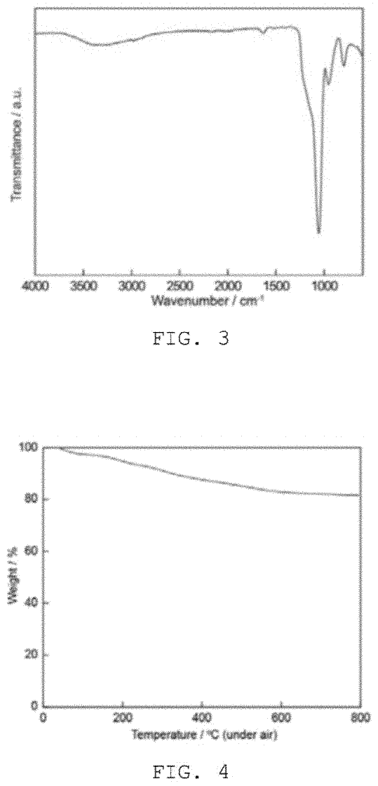

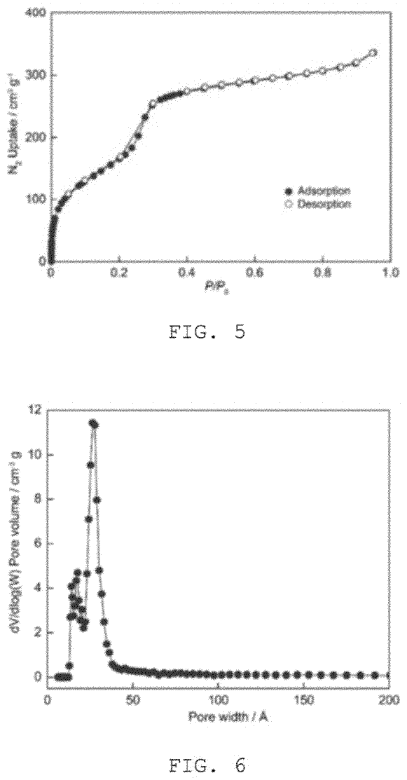

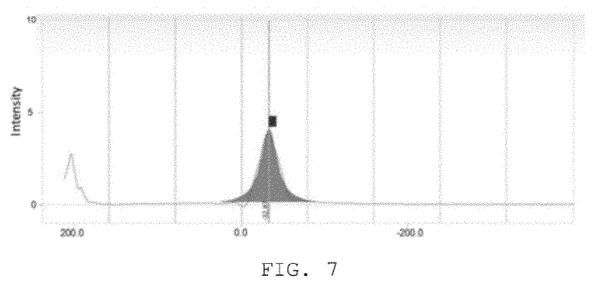

FIG. 1 shows a scanning electron microscopy (SEM) image of the nanoparticles of Example 1 (Gd-MSN). As shown in this figure, the nanoparticles are prepared such that the nanoparticles can have nearly uniform size and shape. FIG. 2 shows a transmission electron microscopy (TEM) image of the nanoparticles. An average particle size of 139 nm was determined from the analysis of the TEM image. FIG. 3 shows a measurement result of FT-IR, in which absorption was observed at 1050 cm⁻¹, indicating a typical band of mesoporous silica. FIG. 4 shows a measurement result of thermal gravitational analysis (TGA), and the TGA chart is involved to a surface modification (phosphonate group) applied to the nanoparticles. FIG. 5 shows a result of nitrogen adsorption/desorption measurement, and based on this result, a graph of the pore size distribution as shown in FIG. 6 was obtained (in FIG. 6, the horizontal axis is represented in angstrom). Based on the pore size distribution, the pore size was calculated to be 3.5 nm. The mesoporous nanoparticles of Comparative Example 1 containing no gadolinium also had the same pore size as described above. As shown in FIG. 7, the zeta potential was −32.87 mV. Accordingly, negative electrification was suggested.

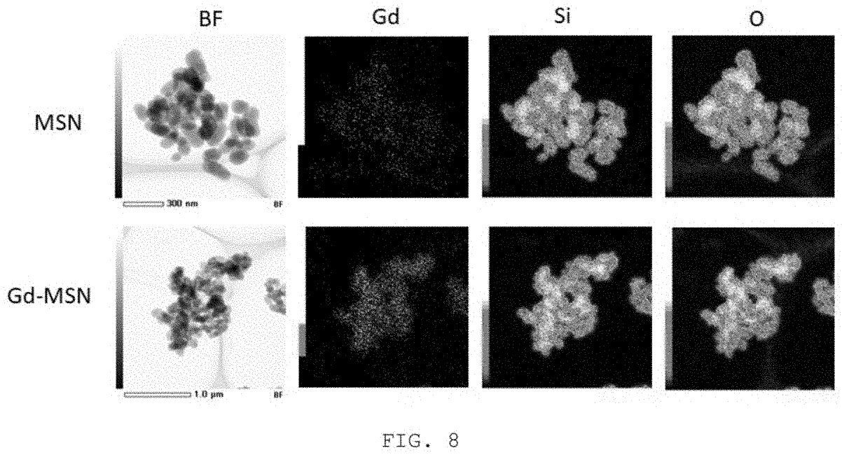

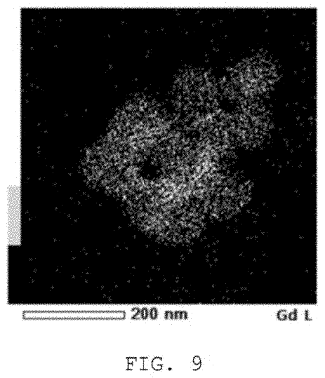

FIG. 8 shows scanning transmission electron microscopy-energy dispersive X-ray (STEM-EDX) mapping images of the nanoparticles of Comparative Example 1 (MSN) and the nanoparticles of Example 1 (Gd-MSN). The element composition of substance and the distribution of elements in the substance can be identified by mapping technology. The images indicated by “BF” are a bright field image, and the images indicated by “Gd”, “Si” and “O” are the mapping results of each of elements. As shown in these figures, the distribution of Gd signal was detected in the Gd-MSN of Example 1, but not in the MSN of Comparative Example 1 (a slightly white portion is considered to be noise). In contrast, Si and O signals were detected in both nanoparticles. This is because Si and O are components of the Si—O—Si framework of the nanoparticles. In the Gd-MSN of Example 1, based on the intensity of the signal, it was estimated that the range occupied by Gd on the nanoparticle was about 1 to 2% relative to the Si. Inductively coupled plasma atomic emission spectroscopy (ICP-AES) analysis showed that the amount of gadolinium is 0.08 mg per 1 mg of MSN.

The stability of bond with gadolinium was investigated with respect to the nanoparticles of Example 1 (Gd-MSN).

The nanoparticles (Gd-MSN) were added to a weakly acidic aqueous solution (nitric acid aqueous solution) prepared and adjusted at pH 5.5, 6.0 or 6.5, and incubated for 2 hours. Then, an amount (concentration) of gadolinium in the aqueous solution was determined by ICP-AES analysis. As a control, the nanoparticles without treatment of the acidic aqueous solution was used. The results are shown in Table 2.

As shown in Table 2, gadolinium was stably bound to MSN even after the treatment with acidic aqueous solution.

TABLE 2

| Treatment | Amount of Gd on MSN (ppm) |
|---|---|
| Control | 1.04 |
| pH 5.5 | 1.03 |
| pH 6.0 | 0.95 |
| pH 6.5 | 0.96 |

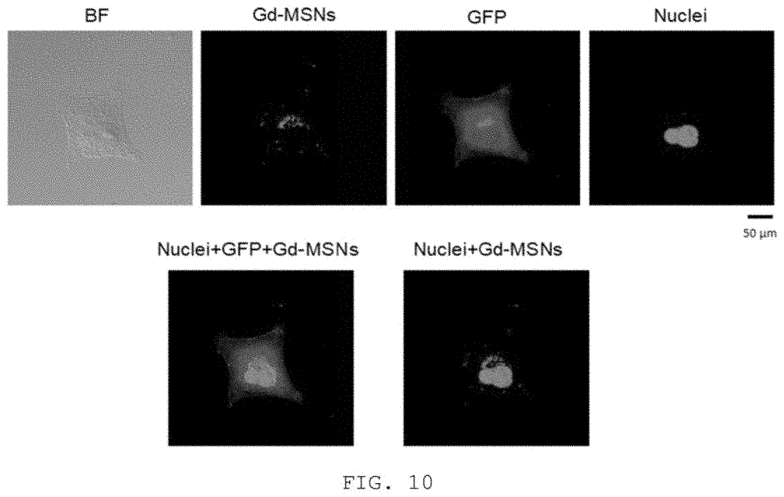

Further, the nanoparticles (Gd-MSN) were mixed with water, ultrasonically treated for 30 minutes, and then STEM-EDX analysis was performed. The results are shown in FIG. 9.

The STEM-EDX image of FIG. 9 shows the presence of Gd. As shown in this image, gadolinium was stably bound to MSN even after sonication.

### Example 3

### Preparation of Iodine-Containing Mesoporous Silica Nanoparticles (I-MSN)

A mixture of cetyltrimethylammonium bromide (CTAB, 98%, Sigma-Aldrich) (250 mg), 8 M NaOH aqueous solution (219 μL) and water (120 mL) was vigorously stirred at 80° C. to prepare a CTAB solution. Whereas, rhodamine B isothiocyanate (RITC, Sigma-Aldrich) (2.5 mg) was dissolved in ethanol (5 mL), and 3-aminopropyltriethoxysilane (APTS, 99%, Wako) (6 μL) was added thereto, and the mixture was then mixed by stirring for 30 minutes at room temperature to prepare a RITC-APTS solution. Then, tetraethyl orthosilicate (TEOS, also known as tetraethoxysilane, 95%, Wako) (1.2 mL) was mixed with the above RITC-APTS solution, and then the mixture solution was added dropwise to the CTAB solution. Then, (3-iodopropyl)trimethoxysilane (0.5 mL) was added thereto, and the mixture was stirred for 15 minutes. To the mixture, 3-(trihydroxysilyl)propyl methylphosphonate monosodium aqueous solution (50%) (315 μL) was then added, and the mixture was stirred. The resulting solid product was collected by centrifugation, and washed three times with ethanol. The solid product was refluxed in a mixed solution of concentrated HCl solution (2.3 mL) and ethanol (60 mL). Accordingly, the CTAB as template was removed. Then, the solid product was centrifuged, washed twice with ethanol, and dried overnight. As a result, iodine-containing mesoporous silica nanoparticles (I-MSN) were obtained.

The obtained nanoparticles each had a diameter of 100 nm, and were uniform particles. The content of iodine atom was 0.033 mg per 1 mg of nanoparticles.

Here, in the above preparation, the rhodamine B labeling was performed for analysis and experiments. However, it is naturally possible to omit the rhodamine B labeling to prepare iodine-containing nanoparticles (I-MSN).

Next, tests of treating cancers, or inhibiting the growth or proliferation of cancers using the nanoparticles of the present invention obtained as described above, and test results thereof are described below.

### Test Example 1

### Uptake of Gd-MSN into Cancer Cells

As cancer cells, human ovarian cancer cells OVCAR8 expressing green fluorescent protein (GFP) were used. The cancer cells OVCAR8 were incubated in RPMI1640 medium supplemented with 10% inactivated FBS and 1% penicillin/streptomycin on a 100 mm culture dish. A predetermined amount of nanoparticles (Gd-MSN) obtained in Example 1 were added to the culture medium, and the cells were incubated for 24 hours at 37° C. in CO₂ incubator. Then, the culture medium was removed, and the cells were washed. The cells were observed through confocal microscopy.

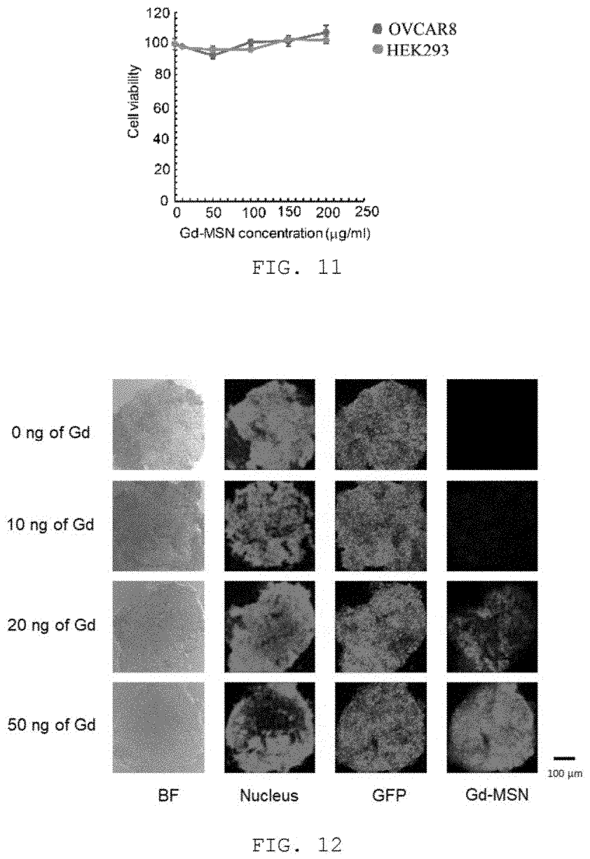

FIG. 10 shows a result of microscopic observation. The image indicated by “BF” shows a bright field image, the image indicated by “Gd-MSNs” shows the result of fluorescence development (red), the image indicated by “GFP” shows the result of fluorescence development (green), and the image indicated by “Nuclei” shows the result of Hoechst dye staining (blue). Further, the images of “Nuclei+GFP+Gd-MSNs” and “Nuclei+Gd-MSNs” show the results of superimposing the respective images. As shown in this figure, the fluorescence development (red) of the nanoparticles is detected just outside the cell nuclei, and is localized to one area of the nuclei. This result suggests that Gd-MSN nanoparticles are efficiently taken into the nucleus of cancer cells.

### Test Example 2

### Safety of Nanoparticles (Gd-MSN) of the Present Invention

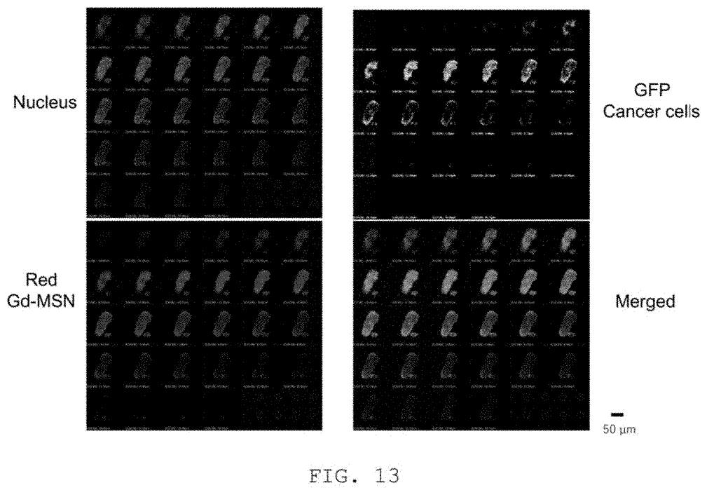

The potential cytotoxicity of the nanoparticles of Example 1 (Gd-MSN) was investigated as follows. The nanoparticles of Example 1 (Gd-MSN) at various amount were incubated with human embryonic kidney HEK293 cells or ovarian cancer OVCAR8 cells. FIG. 11 shows a result of the cytotoxicity test. As shown in FIG. 11, no toxicity was shown up to a concentration of 200 μg/ml.

### Test Example 3

### Uptake of Gd-MSN into Tumor Spheroids

As cancer cells, human ovarian cancer cells OVCAR8 expressing green fluorescent protein (GFP) were used, and tumor spheroids (hereinafter, also referred to as “spheroids”) were prepared from these cancer cells. The cancer cells OVCAR8 were incubated in RPMI1640 medium supplemented with 10% inactivated FBS and 1% penicillin/streptomycin on a 100 mm culture dish. For spheroid formation, 1.0×10⁴ of OVCAR8 cells were inoculated on PrimeSurface 96U culture plate (MS-9096U, Sumitomo Bakelite Co., LTD.). The OVCAR8 cells were cultured for 7 days at 37° C. in CO₂ incubator. Here, since the cells could not adhere to the hydrophilic surface of the plate, the cells were gathered at the bottom of the well, where three-dimensional spheroids were formed. Accordingly, spheroids having a diameter of about 100 to 200 μm were obtained.

Next, the nanoparticles (Gd-MSN) obtained in Example 1 were added to the spheroids, and the spheroids were incubated for 24 hours at 37° C. in CO₂ incubator. The addition amount of nanoparticles (Gd-MSN) was an amount in which an amount by weight of Gd atoms was 10 ng, 20 ng, 50 ng, or 0 ng (i.e. “control”: no addition). After incubation, spheroids were collected in an eppendorf tube, and centrifuged at 1500 rpm for 5 minutes. The supernatant was removed, and the spheroids were washed with ice-cold PBS, centrifuged at 1500 rpm for 5 minutes, and fixed overnight with 4% paraformaldehyde at 4° C. The spheroids were washed with ice-cold PBS, and were treated with 99.8% methanol for 30 minutes at −80° C. The spheroids were washed, and then stained with Hoechst 33258 solution for 30 minutes in dark room. The nanoparticles can be detected by rhodamine B labeling (red fluorescence), and the cell nucleus can be detected by Hoechst dye staining (blue development) and by GFP expression (green fluorescence). The spheroid sample obtained as described above was observed with a confocal microscopy.

FIG. 12 shows images of spheroids observed by microscopy. The images indicated by “BF” show the bright field image, the images indicated by “Nucleus” show the observation result of Hoechst stain (blue), the images indicated by “GFP” show the observation result of fluorescence development (green), the images indicated by “Gd-MSN” show the observation result of fluorescence development (red). As shown in this figure, in “Gd-MSN”, the larger is the amount of Gd, the wider is the range of fluorescence and the higher is the intensity. Thus, the amount of nanoparticles (Gd-MSN) uptake depends on the amount of nanoparticles used for incubation with spheroids.

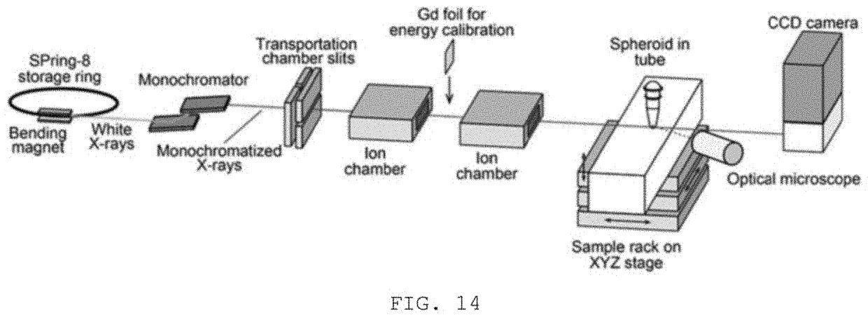

FIG. 13 shows a further result of confocal microscopic observation. This figure shows images at various focal planes. The group of images indicated by “Nucleus” shows the observation result of Hoechst stain (blue), the group of images indicated by “GFP” shows the observation result of fluorescence development (green), the group of images indicated by “Gd-MSN” shows fluorescence development (red). Further, the images indicated by “Merged” show the result obtained by superimposing the above three images. According to this figure, the fluorescence development (red) of the nanoparticles (Gd-MSN) overlaps well with the fluorescence development (green) of GFP, and these overlaps are observed in every focal plane. Accordingly, it is confirmed that the nanoparticles are evenly distributed within the spheroids. It is also confirmed that the nanoparticles have an excellent permeability to spheroids.

### Test Example 4

### Monochromatic X-Ray Irradiation to Gd-MSN

### Setup of Monochromatic X-Ray Irradiation Device

Monochromatic X-ray irradiation was carried out at a beamline BL14B1 of the large synchrotron radiation facility SPring-8 in Sayo-cho, Sayo-gun, Hyogo Prefecture, Japan. FIG. 14 shows an outline of setup of the irradiation device. First, white X-rays generated from a bending electromagnet of SPring-8 were led to a double-crystal fixed-exit spectroscope (monochromator) with silicon 311 crystals to generate a single-energy X-ray beam (monochromatic X-ray). The SPring-8 storage ring was operated in top-up mode with a constant storage ring current of 100 mA, in which X-ray intensity fluctuations in time were negligible. Under these conditions, the irradiation experiments were carried out. The X-ray beam was shaped appropriately by using transport channel (TC) slits consisting of horizontal and vertical slits. The X-ray beam size was 1.4 mm (height)×1.4 mm (width) at the sample position. This size is enough to cover the tumor spheroids that have the dimension of 0.4 mm×0.4 mm. The X-ray beam intensities were monitored by two ion chambers placed on the optical axis during the experiment. The transmitted X-rays were monitored with CCD camera to adjust the sample position. FIG. 15 shows an outline of the spheroid sample place. The spheroids are set at the bottom of a tube that is placed in a sample rack.

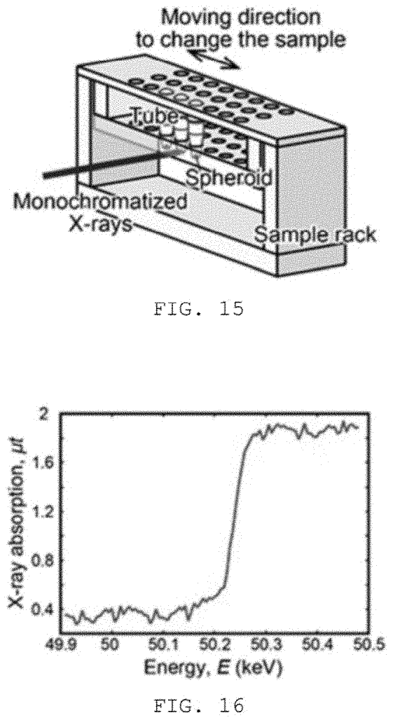

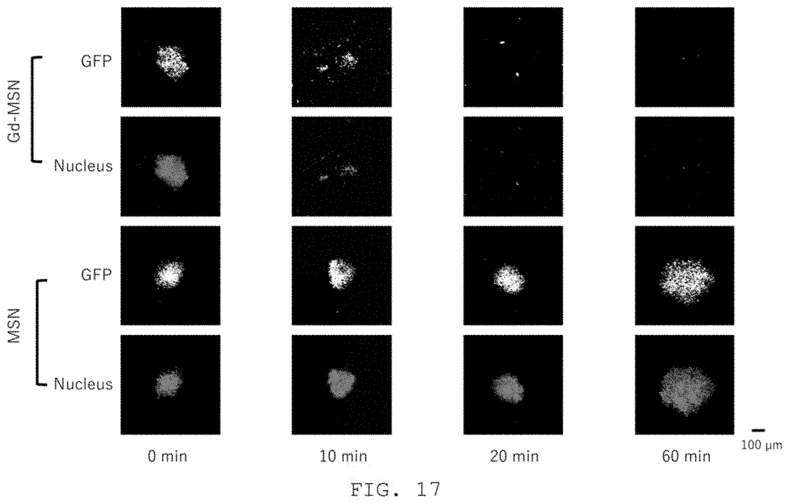

To tune the energy of incident X-rays to be used in the experiment, an X-ray absorption profile of gadolinium was measured at first. A thin foil of gadolinium (80 μm in thickness) was irradiated with monochromatic X-rays having energy level of exact or around K-edge absorption of gadolinium, and X-ray absorption was investigated by the amount of X-rays that were transmitted through the foil. The X-ray absorption “μt” is determined from an equation of μt=−log (I/I₀). Here, “μ” represents a linear absorption coefficient of gadolinium, “t” represents a thickness of gadolinium foil, “I” represents a transmitted X-ray intensity, and “I₀” represents an incident X-ray intensity. FIG. 16 shows a result of the X-ray absorption profile of gadolinium. As shown in this figure, the amount of X-ray absorption was abruptly increased at about 50.2 keV. It was confirmed that the energy of 50.25 keV, which is an intermediate value between the minimum and maximum absorption amounts, corresponds to the K-shell electron excitation energy of gadolinium. Accordingly, monochromatic X-ray having an energy of 50.25 keV were mainly used for the X-ray irradiation to spheroids.

### Test Example 5

### Monochromatic X-ray Irradiation to Spheroids

Nanoparticles and spheroids were incubated in the same manner as in Test Example 3 to obtain spheroids which took in the nanoparticles. The spheroids were placed in a tube, and the tube was placed in a sample rack of the X-ray irradiation device. The sample rack is located such that it can be moved on a XYZ stage, which enables an experimenter to move the sample on the optical axis for X-ray irradiation without entering the experimental hatch. Once the X-ray irradiation to one sample is completed, the sample rack is configured to be moved such that the irradiation position is automatically moved to the next sample, and a series of X-ray irradiations can be performed automatically (See FIG. 15). The sample position was checked before irradiation by using optical microscope and laser. It was also monitored by CCD camera during X-ray irradiations. Here, energies of X-rays were so high that neither the comparative control for absorption of the spheroids nor tubes could be observed by the CCD camera. Refraction-enhanced X-ray images of the tubes were obtained to monitor the sample positions. The photon flux at the sample position was calculated to be 3.11×10⁸ (photons/sec) using SPECTRA code28.

The spheroids prepared as described above were irradiated with monochromatic X-rays for a predetermined time. After X-ray irradiation, the spheroids were incubated in a CO₂ incubator at 37° C. for three days, and the spheroids were then observed under a confocal microscopy (by visible and fluorescent). Further, for comparison, the same operation was performed on the nanoparticles of Comparative Example 1 (MSN without Gd).

### Test Example 6

### Effect of Irradiation Time

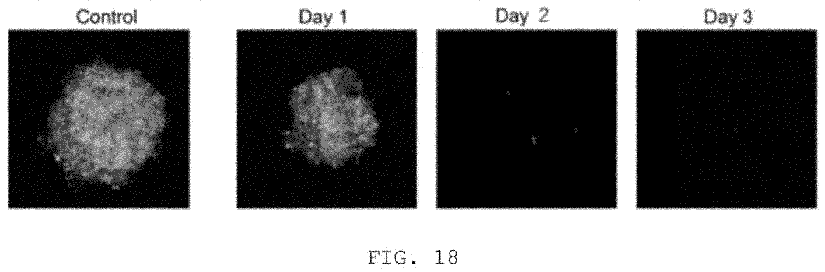

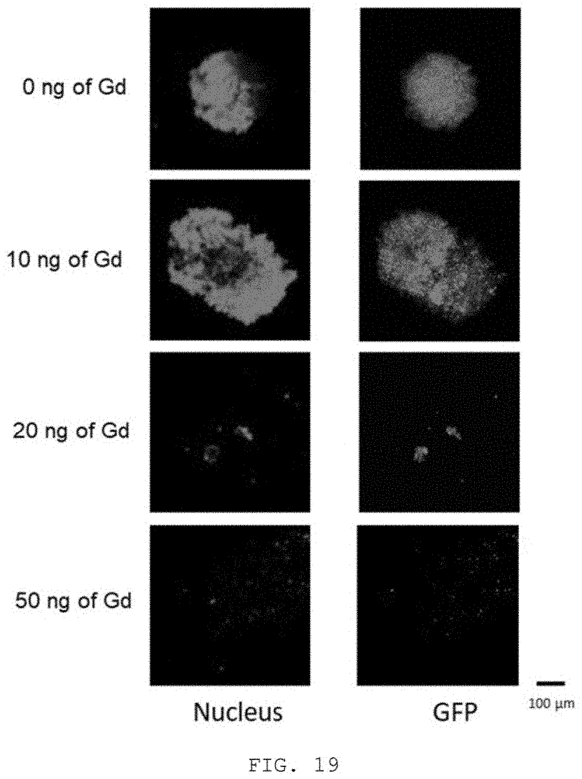

In accordance with Test Example 5, a relationship between X-ray irradiation time and cell destruction was investigated. FIG. 17 shows observation results of the spheroids obtained by irradiating the spheroids with monochromatic X-rays (50.25 keV) at different irradiation times. The two rows indicated by “Gd-MSN” show the results of the spheroids incubated with the nanoparticles of Example 1 (Gd-MSN), and the two rows indicated by “MSN” shows the results of the spheroids incubated with the nanoparticles of Comparative Example 1 (MSN). Images indicated by “GFP” show the fluorescent development (green) of GFP-expressing cancer cells, and images indicated by “Nucleus” show the coloration of cell nucleus (blue) stained with Hoechst dye. In this figure, the results are shown in case that the irradiation time is 0 minute (that is, no irradiation), 10 minutes, 20 minutes, or 60 minutes. As shown in this figure, the spheroids incubated with Gd-MSN were broken up into pieces after 10 minutes of X-ray exposure, and no more spheroid pieces were observed after 60 minutes of X-ray exposure. In contrast, spheroids incubated with MSN were not broken even after 60 minutes of X-ray exposure. Further, the changes over time in cell destruction after X-ray irradiation was investigated (GFP color development), and accordingly, it was confirmed that as shown in FIG. 18, spheroid destruction did not occur immediately after irradiation, but the effect of X-ray irradiation (cell destruction) occurred after incubation for two days or more.

### Test Example 7

### Effect of Amount of Gd-MSN

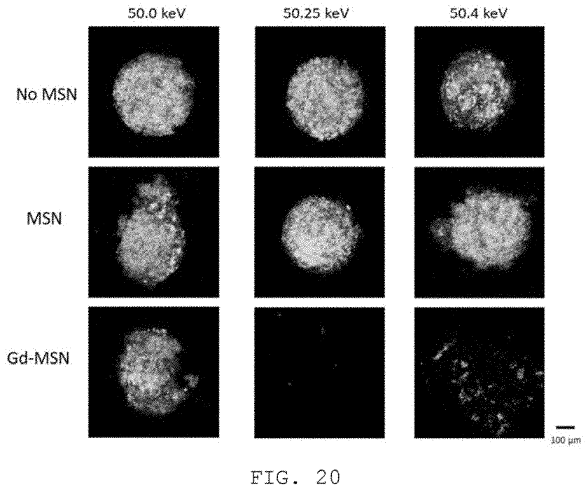

In accordance with Test Example 5, a relationship between an amount of nanoparticles (Gd-MSN) and cell destruction was investigated. FIG. 19 shows a result of the spheroids obtained by irradiating the spheroids with monochromatic X-rays (50.25 keV) for the predetermined time at different amount of nanoparticles (Gd-MSN). As shown in this figure, the spheroids incubated with nanoparticles (Gd-MSN) containing 50 ng of Gd were completely disintegrated after X-ray irradiation, whereas in the case of spheroids incubated with nanoparticles (Gd-MSN) containing 20 ng of Gd, some fragments of the spheroids were able to be observed after the X-ray irradiation. In addition, the spheroids incubated with nanoparticles (Gd-MSN) containing 10 ng of Gd retained their structure even after the irradiation. Thus, the greater was the uptake of Gd-MSN, the higher was the degree of cell destruction.

### Test Example 8

### Effect of Monochromatic X-Ray Energy

In accordance with Test Example 5, a relationship between monochromatic X-ray energy and cell destruction was investigated. FIG. 20 shows a result of the spheroids obtained by irradiating the spheroids with monochromatic X-rays of different energies. Gd-MSN containing 50 ng of Gd was used, and the X-ray irradiation time was 20 minutes. The energy of monochromatic X-rays was 50.0 keV, 50.25 keV, or 50.4 keV. Accordingly, the dependency of X-ray energy on the spheroid destruction was investigated. For comparison, the same operation was performed on spheroids that were incubated without nanoparticles (indicated by “No MSN”) and spheroids that were incubated with the nanoparticles of Comparative Example 1 (MSN, containing no Gd) (indicated by “MSN”). As shown in this figure, with respect to “Gd-MSN”, almost no spheroid destruction was observed with X-rays of 50.0 keV, whereas the spheroids were completely destroyed with X-rays of 50.25 keV. In the case of X-rays with 50.4 keV, the destruction was also observed, however, spheroid residues were detected. Thus, the destruction effect was most excellent in the case of 50.25 keV. This energy corresponds to the K-shell electron excitation energy of gadolinium (Gd), which suggests that cells are destroyed by the Auger effect.

### Test Example 9

### Iodine-containing Mesoporous Silica Nanoparticles (I-MSN) for Cancer Cells

In Test Example 1 described above, a test of uptake into cancer cells is carried out in the same manner as in Test Example 1 except that I-MSN is used instead of Gd-MSN. According to this test, it is confirmed that I-MSN nanoparticles are detected just outside cell nucleus, and are taken into the cancer cell nucleus efficiently.

Further, in Test Example 2, a test is carried out in the same manner as in Test Example 2 except that I-MSN is used instead of Gd-MSN, and accordingly, safety of I-MSN is confirmed.

In Test Example 3, a test of uptake into spheroids is carried out in the same manner as in Test Example 3 except that I-MSN is used instead of Gd-MSN. According to this test, it is shown that the nanoparticles are evenly distributed within the spheroids and that the nanoparticles have excellent permeability to the spheroids.

### Test Example 10

### Monochromatic X-Ray Irradiation to I-MSN

Monochromatic X-ray irradiation set-up is performed in accordance with Test Example 4 as described above, and thus, it is confirmed that the K-shell electron excitation energy of iodine atom corresponds to 33.18 keV.

In Test Example 5 described above, spheroids are irradiated with monochromatic X-rays in the same manner as in Test Example 5 except that I-MSN is used instead of Gd-MSN and the energy of monochromatic X-rays is changed to the energy to be used for iodine atom (33.18 keV). It is confirmed spheroids are destroyed after X-ray irradiation. That is, the X-ray of 33.00 keV shows almost no destruction of spheroids, whereas the X-ray of 33.18 keV shows complete destruction of spheroids. In the case of the X-ray of 33.40 keV, destruction is also observed, but spheroid residues are detected. Thus, the destruction effect is most excellent in the case of X-rays of 33.18 keV.

1. A nanoparticle comprising a compound which comprises
a porous silica; and
at least one high-Z atom selected from a group consisting of a gadolinium atom, an iodine atom, a gold atom, a silver atom and a platinum atom.

2. The nanoparticle according to claim 1, wherein the high-Z atom is present on a surface of or inside the porous silica.

3. The nanoparticle according to claim 1, wherein the high-Z atom or a group containing the high-Z atom is chemically bound to the porous silica.

4. The nanoparticle according to claim 1, wherein the porous silica contains a binding group which is bound to the high-Z atom or the group containing the high-Z atom.

5. The nanoparticle according to claim 4, wherein the binding group which is bound to the high-Z atom or the group containing the high-Z atom is selected from an amino group, a carboxy group, or a hydroxy group.

6. The nanoparticle according to claim 1, wherein the porous silica contains a group for inhibiting an aggregation of nanoparticles.

7. The nanoparticle according to claim 6, wherein the group for inhibiting the aggregation of nanoparticles is selected from a phosphonate group, a sulfonate group, or a carboxylate group.

8. The nanoparticle according to claim 1, wherein the nanoparticle has a particle size of 40 to 400 nm in diameter.

9. The nanoparticle according to claim 1, wherein the porous silica is a mesoporous silica.

10. The nanoparticle according to claim 9, wherein the mesoporous silica is a biodegradable mesoporous silica.

11. The nanoparticle according to claim 1, wherein the nanoparticle is subjected to an irradiation with X-ray capable of exciting a K-shell electron of the high-Z atom.

12. The nanoparticle according to claim 11, wherein the X-ray is monochromatic X-ray or characteristic X-ray.

13. A pharmaceutical composition for radiotherapy which comprises the nanoparticle according to claim 1 and a pharmaceutically acceptable carrier.

14. The pharmaceutical composition for radiotherapy according to claim 13, wherein the pharmaceutical composition is subjected to an irradiation with X-ray that is a radiation for irradiation.

15. The pharmaceutical composition for radiotherapy according to claim 13, wherein the pharmaceutical composition is used for treating solid cancer, or inhibiting a growth or proliferation of solid cancer.

16. The pharmaceutical composition for radiotherapy according to claim 15, wherein the solid cancer is selected from a brain tumor, a lung cancer, an ovarian cancer, a digestive system cancer, an osteosarcoma, or a head and neck cancer.

17. A method for treating solid cancer or inhibiting a growth or proliferation of solid cancer, wherein the method comprises subjecting the nanoparticle according to claim 1 which is taken into a body of subject to a X-ray irradiation to destroy a cancer cell.

18. A method for preparing the nanoparticle according to claim 1, which comprises at least one step selected from
(a) a step of adding a substance containing iodine atom to a precursor substance for forming porous silica to incorporate the iodine atom inside a structure of the porous silica; and
(b) a step of adding a substance which contains a binding group for binding to a compound containing gadolinium atom or a precursor group of the binding group to a precursor substance for forming porous silica such that the porous silica can contain the binding group for binding to the compound containing gadolinium atom or the precursor group of the binding group; and then chemically bonding the compound containing gadolinium atom to the porous silica via the binding group for binding to the compound containing gadolinium atom or the precursor group of the binding group.

19. The method according to claim 17, wherein the solid cancer is selected from a brain tumor, a lung cancer, an ovarian cancer, a digestive system cancer, an osteosarcoma, or a head and neck cancer.
